(19) United States
(12) Patent Application Publication
(10) Pub. No.: US 2009/0048365 A1
(43) Pub. Date: Feb. 19, 2009

(54) Odor-Absorbing Capsule Particle Composition and Methods Therein

(76) Inventors: Joseph Brain, Locust, NJ (US); Jeffrey Bonn, Emerson, NJ (US); Judith Kerschner, Hawthorne, NJ (US); Timothy Young, Middletown, NJ (US); Ronald Swayze, Beachwood, NJ (US)

Correspondence Address: INTERNATIONAL FLAVORS & FRAGRANCES INC., 521 WEST 57TH ST, NEW YORK, NY, 10019, US

(21) Appl. No.: 11/840,354

(22) Filed: Aug. 17, 2007

(51) Int. Cl. A61L 9/04

(52) U.S. Cl. 523/102

(57) ABSTRACT

An odor-absorbing capsule particle composition and methods of eliminating malodor from hard and soft surfaces and the surrounding vapor phase is provided.

### FIELD OF THE INVENTION

The present invention relates to the absorption of malodor molecules with capsule particles that may comprise active materials that are encapsulated with a polymeric material.

### BACKGROUND OF THE INVENTION

The need for imparting substantive fragrances to, and removing or covering a perceived malodor from solid or semi-solid surfaces including fabric surfaces such as surfaces of articles of clothing being washed, the human epidermis, hair follicles and solid surfaces such as tile kitchen counters has been, over the past century, well-recognized in the prior art. Attempts at fulfilling these needs using various delivery system have been disclosed in the prior art.

In order to enhance the effectiveness of the malodor reduction materials for the user, various technologies have been employed to enhance the delivery of the malodor reducing materials at the desired time.

Nothing in the prior art discloses or suggests a method for imparting substantive fragrances to, and/or absorbing perceived malodors from solid or semi-solid surfaces using encapsulated materials where the polymers which compose the encapsulated material are compatible with (a) malodor substances absorbable by the encapsulated material and/or (b) malodor materials are absorbable into the encapsulated material.

### SUMMARY OF THE INVENTION

The present invention relates to an odor-absorbing composition comprising an effective amount of capsule particle composition to absorb malodors, wherein the capsule particles are comprised of an active material which is encapsulated by an encapsulating polymer.

In a further embodiment of the invention, a method of absorbing malodor from hard surfaces, soft surfaces and the surrounding air is provided comprising applying an effective amount of the capsule particle formulation to the desired surface.

According to one embodiment, the product containing the capsule particle composition may be a “leave-on” product such as a fabric refresher spray, hard-surface cleaning product, carpet cleaning spray, a deodorant and antiperspirant, etc.

The product containing the capsule particle composition may also be a “wash off” product such as a fabric conditioner, laundry detergents, a personal cleaning product, shampoo and conditioners, liquid, gel and bar soap, and hard-surface cleaning products.

In a further embodiment a method to absorb malodors inherent in a products, such as detergents, soap bars, shampoo and conditioners, hair color and dyes, antiperspirants and roll-on deodorants is provided.

In yet a further embodiment, a formulation containing an effective amount of the capsule particles may be provided in a spray form, such as an aerosol, pump or trigger spray for removing malodor from the air and surrounding hard and soft surfaces.

These and other embodiments of the present invention will become apparent upon referring to the detailed description of the invention.

### DETAILED DESCRIPTION OF THE INVENTION

The capsule particle composition comprises an active material encapsulated by a crosslinked network of polymers. The capsule particle composition is capable of absorbing malodour from both hard surfaces and soft surfaces and the surrounding vapor phase. Malodor ingredients that the capsule composition of the present invention are capable of reducing include, but are not limited to, pyridine, furfural, isovaleric acid, heptanal, hexanoic acid, and dibutyl sulfide.

The capsule particle composition of the present invention provides a measurable benefit in the actual reduction of these malodour ingredients in the vapor phase, to which the examples of the present invention demonstrate. There is also a large benefit in the fact that capsules can carry a fragrance which can be designed to cover malodor and reduce the perception malodor.

In general, the present compositions can comprise microcapsules at a wide variety of levels. Microcapsules are typically included in the present compositions at a level of from about 0.001% to about 99.9%, preferably from about 0.005% to about 50%, and more preferably from about 0.01% to about 20%, by weight of the composition. When the compositions are aqueous liquid compositions (especially non-aerosol compositions) to be sprayed onto surfaces, such as fabrics, the compositions will preferably comprise less than about 1%, preferably less than about 0.9%, more preferably less than about 0.5%, and even more preferably less than about 0.2%, by weight of the composition, of microcapsules. If the level of microcapsules is too high, the compositions may leave a visible residue on the surface being treated. In addition, if the surface is fabric and the level of microcapsules is too high, the fabric appearance may be altered. Furthermore, if the active material is perfume and the level of microcapsules is too high, the initial perfume “burst” when the product is sprayed onto the surface may be unpleasant to the consumer, since the force of the spray tends to rupture some of the microcapsules.

The term vapor phase will be used to refer to the air above a hard surface or a soft surface.

The term hard surface will be used to refer to solid non-porous surfaces such as counter tops, glass, wood, tile and flooring.

The term soft surfaces herein will be used to refer to carpeting, upholstery and other fabrics that are porous as opposed to hard such as bed linens, bath linens, table clothes and also hair and skin surfaces.

The term “malodor” as used herein refers to a distinctive odor that is offensively unpleasant smell that may be associated with or emanating from animal or plant waste such as that caused by the presence of compounds and by products including, but not limited to, volatile, odorous, organic acids as well as sulfur and nitrogen containing compounds.

As described herein, the odor-absorbing composition of the present invention is well suited for use in a variety of well-known consumer products such as liquid, powder and gel laundry detergent and fabric softeners, liquid, gel and powder dish detergents, automatic dish detergents, as well as hair shampoos and conditioners. These products employ surfactant and emulsifying systems that are well known. For example, fabric softener systems are described in U.S. Pat. Nos. 6,335,315, 5,674,832, 5,759,990, 5,877,145, 5,574,179; 5,562,849, 5,545,350, 5,545,340, 5,411,671, 5,403,499, 5,288,417, 4,767,547, 4,424,134. Liquid dish detergents are described in U.S. Pat. Nos. 6,069,122 and 5,990,065; automatic dish detergent products are described in U.S. Pat. Nos. 6,020,294, 6,017,871, 5,968,881, 5,962,386, 5,939,373, 5,914,307, 5,902,781, 5,705,464, 5,703,034, 5,703,030, 5,679,630, 5,597,936, 5,581,005, 5,559,261, 4,515,705, 5,169,552, and 4,714,562. Laundry detergents which can use the present invention include those systems described in U.S. Pat. Nos. 5,929,022, 5,916,862, 5,731,278, 5,565,145, 5,470,507, 5,466,802, 5,460,752, 5,458,810, 5,458,809, 5,288,431,5,194,639, 4,968,451, 4,597,898, 4,561,998, 4,550,862, 4,537,707, 4,537,706, 4,515,705, 4,446,042, and 4,318,818. Shampoo and conditioners that can employ the present invention include U.S. Pat. Nos. 6,162,423, 5,968,286, 5,935561, 5,932,203, 5,837,661, 5,776,443, 5,756,436, 5,661,118, 5,618,523, 5,275,755, 5,085,857, 4,673,568, 4,387,090 and 4,705,681.

Rheology modifiers should be selected carefully to insure compatibility with the deposition agents. Preferred are nonionic, cationic and amphoteric thickeners, such as modified polysaccharides (starch, guar, celluloses), polyethylene imine (Lupasol WF, BASF Corporation), acrylates (Structure Plus, National Starch and Chemical Company) and cationic silicones.

In order to provide the capsule particle composition in a dry form, it is preferable that the materials be dried using drying techniques well known in the art. In a preferred embodiment the materials are spray dried under the appropriate conditions. The spray dried particles may also be sized to provide for consistent particle size and particle size distribution. One application in which it would be advantageous to include dry particles of the present invention would be incorporated in a powdered laundry detergent. Alternatively wet capsule particle compositions may be absorbed onto suitable dry powders to yield a flowable solid suitable for dry product use.

The present invention encompasses the method of eliminating the perception of malodor by washing a fabric and/or fabric articles, which may be selected from but not limited to, clothes, curtains, drapes, upholstered furniture, carpeting, bed linens, bath linens, tablecloths, sleeping bags, tents, car interior, etc., also hard-surfaces such as floors, countertops, bathroom tile with a fabric conditioner, hard surface cleaner, or detergent containing an effective amount of the capsule particles composition.

The present invention encompasses the method of spraying an effective amount of capsule particle composition onto household surfaces. Preferably said household surfaces are selected from the group consisting of countertops, cabinets, walls, floors, upholstery, curtains, bathroom surfaces and kitchen surfaces.

The present invention encompasses the method of spraying a mist of an effective amount of capsule particle composition onto fabric and/or fabric articles. Preferably, said fabric and/or fabric articles include, but are not limited to, clothes, curtains, drapes, upholstered furniture, carpeting, bed linens, bath linens, tablecloths, sleeping bags, tents, car interior, etc.

The present invention encompasses the method of spraying a mist of an effective amount of capsule particle composition onto and into shoes wherein said shoes are not sprayed to saturation.

The present invention encompasses the method of spraying a mist of an effective amount of capsule particle composition onto shower curtains.

The present invention relates to the method of spraying a mist of an effective amount of capsule particle composition onto garbage cans and recycling bins.

The present invention relates to the method of spraying a mist of an effective amount of capsule particle composition into the air to absorb malodor.

The present invention relates to the method of spraying a mist of an effective amount of capsule particle composition into and/or onto major household appliances including, but not limited to: refrigerators, freezers, washing machines, automatic dryers, ovens, microwave ovens, and dishwashers to absorb malodor.

The present invention relates to the method of spraying a mist of an effective amount of capsule particle composition onto cat litter, pet bedding and pet houses to absorb malodor.

The present invention relates to the method of spraying a mist of an effective amount of capsule particle composition onto household pets to absorb malodor. Also, the capsule particles may comprise active ingredients such as flea, tick and insect repellant that can be used on pets.

### Capsule Particles

The capsule particles of the present invention may be prepared according to the process described in the following U.S. Pat. Nos. 2,800,457, 3,870,542, 3,516,941, 3,415,758, 3,041,288, 5,112,688, 6,329,057, and 6,261,483. Another discussion of fragrance encapsulation is found in the Kirk-Othmer Encyclopedia. The present invention also contemplated the encapsulation of active materials which included but is not limited to perfumes, flavoring agents, fungicide, brighteners, antistatic agents, wrinkle control agents, fabric softener actives, hard surface cleaning actives, skin and/or hair conditioning agents, malodour counteractants, antimicrobial actives, UV protection agents, insect repellents, animal/vermin repellants, flame retardants, and the like.

Preferred encapsulating polymers include those formed from melamine-formaldehyde or urea-formaldehyde condensates, as well as similar types of aminoplasts. Additionally, capsules made via the simple or complex coacervation of gelatin are also preferred for use with the coating. Capsules having shell walls comprised of polyurethane, polyamide, polyolefin, polysaccaharide, protein, silicone, lipid, modified cellulose, gums, polyacrylate, polyphosphazines, polystyrene, and polyesters or combinations of these materials are also functional.

The preferred encapsulated polymers of the present invention are selected from, but not limited to, a vinyl polymer, an acrylate polymer, melamine-formaldehyde, urea formaldehyde, amine-containing polymer, amine-generating polymer, aminoplasts, aldehydes, dialdehydes, active oxygen, poly-substituted carboxylic acids and derivatives, inorganic crosslinkers, organics capable of forming azo, azoxy and hydrazo bonds, lactones and lactams, thionyl chloride, phosgene, tannin/tannic acid, polyphenols, free radical crosslinkers, sodium persulfate, azoisobutylnitrile (AIBN) and mixtures thereof.

In one embodiment the crosslinked network of polymers comprises a melamine-formaldehyde:acrylamide-acrylic acid copolymer wherein the mole ratio is in the range of from 9:1 to 1:9, more preferable 5:1 to 1:5 and most preferably 2:1 to 1:2.

A representative process used to prepare the capsule particles of the present invention is disclosed in U.S. Pat. No. 3,516,941 though it is recognized that many variations with regard to materials and process steps are possible. A representative process used for gelatin encapsulation is disclosed in U.S. Pat. No, 2,800,457 though it is recognized that many variations with regard to materials and process steps are possible. Both of these processes are discussed in the context of fragrance encapsulation for use in consumer products in U.S. Pat. Nos. 4,145,184 and 5,112,688 respectively.

In one embodiment of the present invention, the capsule particles may contain materials such as solvents, surfactants, emulsifiers, and the like can be used alone or in addition to the polymers described above to encapsulate the fragrance without departing from the scope of the present invention.

It is understood that the term encapsulated is meant to mean that the active material is substantially covered in its entirety. Encapsulation can provide pore vacancies or interstitial openings depending on the encapsulation techniques employed. More preferably the entire active material portion of the present invention is encapsulated to form capsule particles.

Particle and capsule diameter can vary from about 10 nanometers to about 1000 microns, preferably from about 50 nanometers to about 100 microns and is most preferably from about 2 to about 15 microns. The capsule distribution can be narrow, broad, or multi-modal. Each modal of the multi-modal distributions may be composed of a different type of capsule chemistry.

The weight ratio of the encapsulating polymer to active material is from about 1:25 to about 1:1. Preferred products have had the weight ratio of the encapsulating polymer to active material varying from about 1:10 to about 4:96.

For example, if a capsule blend has 20 weight % fragrance and 20 weight % polymer, the polymer ratio would be (20/20) multiplied by 100(%)=100%.

### Active Ingredients

According to present invention, the capsule particles may contain active ingredients selected from, but not limited to fragrances, solvents such as but not limited to neobee oil, mineral oil, silicon oil, organic oil, hydrophobic solvents, triglyceride oils, fats, waxes, fatty alcohols, diisodecyl adipate and diethyl phthalate, antioxidants, anti-microbial agents, enzymes, fungicides and reactive agents.

In one embodiment, fragrances can be employed in the present invention, the only limitation being the compatibility and ability to be encapsulated by the polymer being employed, and compatability with the encapsulation process used. Suitable fragrances include but are not limited to fruits such as almond, apple, cherry, grape, pear, pineapple, orange, strawberry, raspberry; musk, flower scents such as lavender-like, rose-like, iris-like, and carnation-like. Other pleasant scents include herbal scents such as rosemary, thyme, and sage; and woodland scents derived from pine, spruce and other forest smells. Fragrances may also be derived from various oils, such as essential oils, or from plant materials such as peppermint, spearmint and the like. Other familiar and popular smells can also be employed such as baby powder, popcorn, pizza, cotton candy and the like in the present invention.

A list of suitable fragrances is provided in U.S. Pat. Nos. 4,534,891, 5,112,688 and 5,145,842. Another source of suitable fragrances is found in Perfumes Cosmetics and Soaps, Second Edition, edited by W. A. Poucher, 1959. Among the fragrances provided in this treatise are acacia, cassie, chypre, yclamen, fern, gardenia, hawthorn, heliotrope, honeysuckle, hyacinth, jasmine, lilac, lily, magnolia, mimosa, narcissus, freshly-cut hay, orange blossom, orchids, reseda, sweet pea, trefle, tuberose, vanilla, violet, wallflower, and the like.

As used herein olfactory effective amount is understood to mean the amount of compound in perfume compositions the individual component will contribute to its particular olfactory characteristics, but the olfactory effect of the fragrance composition will be the sum of the effects of each of the fragrance ingredients. Thus the compounds of the invention can be used to alter the aroma characteristics of the perfume composition by modifying the olfactory reaction contributed by another ingredient in the composition. The amount will vary depending on many factors including other ingredients, their relative amounts and the effect that is desired.

The level of active material in the capsule particle varies from about 5 to about 95 weight percent, preferably from about 40 to about 95 and most preferably from about 50 to about 90 weight percent.

As noted above, a variety of solvents may be used alone or in combination with a fragrance to serve to increase the compatibility of the various materials, increase the overall hydrophobicity of the blend, influence the vapor pressure of the materials, or serve to structure the blend.

A common feature of many encapsulation processes is that they require the active material to be encapsulated to be dispersed in aqueous solutions of polymers, pre-condensates, surfactants, and the like prior to formation of the capsule walls. Therefore, materials having low solubility in water, such as highly hydrophobic materials are preferred, as they will tend to remain in the dispersed perfume phase and partition only slightly into the aqueous solution.

In a one embodiment, fragrance materials are employed with Clog P values greater than 1 and preferably greater than 3 will thus result in micro-capsules that contain cores most similar to the original composition, and will have less possibility of reacting with materials that form the capsule shell.

When using fragrances, in order to provide the highest fragrance impact from the fragrance encapsulated capsules deposited on the various substrates referenced above, it is preferred that materials with a high odor-activity be used. Materials with high odor-activity can be detected by sensory receptors at low concentrations in air, thus providing high fragrance perception from low levels of deposited capsules. This property must be balanced with the volatility as described above. Some of the principles mentioned above are disclosed in U.S. Pat. No. 5,112,688.

One measurement of the enhancement of the present invention in delivering the fragrance and other ingredients of the present invention is done by headspace analysis. Headspace analysis can provide a measure of the fragrance material contained on the desired substrate provided by the present invention. The present invention will provide a much higher level of fragrance on the substrate compared to the amount of fragrance deposited on the substrate by conventional means.

### Article of Manufacture

The composition of the present invention can also be used in an article of manufacture comprising said composition plus a spray dispenser. When the commercial embodiment of the article of manufacture is used, it is optional, but preferable, to include the preservative. Therefore, the most basic article of manufacture comprises capsule particle composition, a carrier, and a spray dispenser.

### Spray Dispenser

The article of manufacture herein comprises a spray dispenser. The capsule particle composition is placed into a spray dispenser in order to be distributed onto the fabric. Said spray dispenser is preferably any of the manually activated means for producing a spray of liquid droplets as is known in the art, e.g. trigger-type, pump-type, non-aerosol self-pressurized, and aerosol-type spray means. The spray dispenser herein does not normally include those that will substantially foam the clear, aqueous odor absorbing composition.

The spray dispenser can be an aerosol dispenser. Said aerosol dispenser comprises a container which can be constructed of any of the conventional materials employed in fabricating aerosol containers. The one important requirement concerning the dispenser is that it be provided with a valve member which will permit the clear, aqueous odor absorbing composition contained in the dispenser to be dispensed in the form of a spray of very fine, or finely divided, particles or droplets. The aerosol dispenser utilizes a pressurized sealed container from which the clear, aqueous odor-absorbing composition is dispensed through a special actuator/valve assembly under pressure. The aerosol dispenser is pressurized by incorporating therein a gaseous component generally known as a propellant. Preferred propellants are compressed air, nitrogen, inert gases, carbon dioxide, etc. A more complete description of commercially available aerosol-spray dispensers appears in U.S. Pat. No. 3,436,772. Stebbins, issued Apr. 8, 1969; and U.S. Pat. No. 3,600,325, Kaufman et al., issued Aug. 17, 1971; both of said references are incorporated herein by reference.

Preferably the spray dispenser can be a self-pressurized non-aerosol container having a convoluted liner and an elastomeric sleeve. Said self-pressurized dispenser comprises a liner/sleeve assembly containing a thin, flexible radially expandable convoluted plastic liner of from about 0.010 to about 0.020 inch thick, inside an essentially cylindrical elastomeric sleeve. The liner/sleeve is capable of holding a substantial quantity of odor-absorbing fluid product and of causing said product to be dispensed. A more complete description of self-pressurized spray dispensers can be found in U.S. Pat. No. 5,111,971, Winer, issued May 12, 1992, and U.S. Pat. No. 5,232,126, Winer, issued Aug. 3, 1993; both of said references are herein incorporated by reference. Another type of aerosol spray dispenser is one wherein a barrier separates the odor absorbing composition from the propellant (preferably compressed air or nitrogen), as disclosed in U.S. Pat. No. 4,260,110, issued Apr. 7, 1981, and incorporated herein by reference. Such a dispenser is available from EP Spray Systems, East Hanover, N.J.

More preferably, the spray dispenser is a non-aerosol, manually activated, pump-spray dispenser. Said pump-spray dispenser comprises a container and a pump mechanism which securely screws or snaps onto the container. The container comprises a vessel for containing the aqueous odor-absorbing composition to be dispensed.

The pump mechanism comprises a pump chamber of substantially fixed volume, having an opening at the inner end thereof. Within the pump chamber is located a pump stem having a piston on the end thereof disposed for reciprocal motion in the pump chamber. The pump stem has a passageway there through with a dispensing outlet at the outer end of the passageway and an axial inlet port located inwardly thereof.

The container and the pump mechanism can be constructed of any conventional material employed in fabricating pump-spray dispensers, including, but not limited to: polyethylene; polypropylene; polyethyleneterephthalate; blends of polyethylene, vinyl acetate, and rubber elastomer. A preferred container is made of clear, e.g., polyethylene terephthalate. Other materials can include stainless steel. A more complete disclosure of commercially available dispensing devices appears in: U.S. Pat. No. 4,895,279, Schultz, issued Jan. 23, 1990; U.S. Pat. No. 4,735,347, Schultz et al., issued Apr. 5, 1988; and U.S. Pat. No. 4,274,560, Carter, issued Jun. 23, 1981; all of said references are herein incorporated by reference.

Most preferably, the spray dispenser is a manually activated trigger-spray dispenser. Said trigger-spray dispenser comprises a container and a trigger both of which can be constructed of any of the conventional material employed in fabricating trigger-spray dispensers, including, but not limited to: polyethylene; polypropylene; polyacetal; polycarbonate; polyethyleneterephthalate; polyvinyl chloride; polystyrene; blends of polyethylene, vinyl acetate, and rubber elastomer. Other materials can include stainless steel and glass. A preferred container is made of clear, e.g. polyethylene terephthalate. The trigger-spray dispenser does not incorporate a propellant gas into the odor-absorbing composition, and preferably it does not include those that will foam the odor-absorbing composition. The trigger-spray dispenser herein is typically one which acts upon a discrete amount of the odor-absorbing composition itself, typically by means of a piston or a collapsing bellows that displaces the composition through a nozzle to create a spray of thin liquid. Said trigger-spray dispenser typically comprises a pump chamber having either a piston or bellows which is movable through a limited stroke response to the trigger for varying the volume of said pump chamber. This pump chamber or bellows chamber collects and holds the product for dispensing. The trigger spray dispenser typically has an outlet check valve for blocking communication and flow of fluid through the nozzle and is responsive to the pressure inside the chamber. For the piston type trigger sprayers, as the trigger is compressed, it acts on the fluid in the chamber and the spring, increasing the pressure on the fluid. For the bellows spray dispenser, as the bellows is compressed, the pressure increases on the fluid. The increase in fluid pressure in the trigger spray dispenser acts to open the top outlet check valve. The top valve allows the product to be forced through the swirl chamber and out the nozzle to form a discharge pattern. An adjustable nozzle cap can be used to vary the pattern of the fluid dispensed.

For the piston spray dispenser, as the trigger is released, the spring acts on the piston to return it to its original position. For the bellows spray dispenser, the bellows acts as the spring to return to its original position. This action causes a vacuum in the chamber. The responding fluid acts to close the outlet valve while opening the inlet valve drawing product up to the chamber from the reservoir.

A more complete disclosure of commercially available dispensing devices appears in U.S. Pat. No. 4,082,223, Nozawa, issued Apr. 4, 1978; U.S. Pat. No. 4,161,288, McKinney, issued Jul. 17, 1985; U.S. Pat. No. 4,434,917, Saito et al., issued Mar. 6, 1984; and U.S. Pat. No. 4,819,835, Tasaki, issued Apr. 11, 1989; U.S. Pat. No. 5,303,867, Peterson, issued Apr. 19, 1994; all of said references are incorporated herein by reference.

A broad array of trigger sprayers and finger pump sprayers are suitable for use with the compositions of this invention. These are readily available from suppliers such as Calmar, Inc., City of Industry, Calif.; CSI (Continental Sprayers, Inc.), St. Peters, Mo.; Berry Plastics Corp., Evansville, Ind., a distributor of Guala® sprayers; or Seaquest Dispensing, Cary, Ill.

The preferred trigger sprayers are the blue inserted Guala.RTM. sprayer, available from Berry Plastics Corp., or the Calmar TS800-1A®, TS1300®, and TS-800-2®, available from Calmar Inc., because of the fine uniform spray characteristics, spray volume, and pattern size. More preferred are sprayers with precompression features and finer spray characteristics and even distribution, such as Yoshino sprayers from Japan. Any suitable bottle or container can be used with the trigger sprayer, the preferred bottle is a 17 fl-oz. bottle (about 500 ml) of good ergonomics similar in shape to the Cinch® bottle. It can be made of any materials such as high density polyethylene, polypropylene, polyvinyl chloride, polystyrene, polyethylene terephthalate, glass, or any other material that forms bottles. Preferably, it is made of high density polyethylene or clear polyethylene terephthalate.

For smaller fluid ounce sizes (such as 1 to 8 ounces) a finger pump can be used with canister or cylindrical bottle. The preferred pump for this application is the cylindrical Euromist II® from Seaquest Dispensing. More preferred are those with precompression features.

These and additional modifications and improvements of the present invention may also be apparent to those with ordinary skill in the art. The particular combinations of elements described and illustrated herein are intended only to represent only a certain embodiment of the present invention and are not intended to serve as limitations of alternative articles within the spirit and scope of the invention. All materials are reported in weight percent unless noted otherwise. As used herein all percentages are understood to be weight percent.

All U.S. patents and patent applications cited herein are incorporated by reference as if set forth in their entirety.

### EXAMPLE 1

### Preparation of Capsule Particle Composition

In this example, melamine-formaldehyde capsule slurry (uncoated capsules made by Cellessence International Ltd., West Molesey, Surrey, UK) that contains approximately 32% by weight of the fragrance and 57% by weight of water was used. To make the capsule slurry, a copolymer of poly acrylamide and acrylic acid was first dispersed in water together with a methylated melamine-formaldehyde resin. Fragrance was then added into the solution with high speed shearing to form small droplets.

### EXAMPLE 2

### Effectiveness of these Encapsulated Materials Toward Malodor Reduction

An analysis of the malodor present in the headspace with and without the encapsulated material was conducted. For this experiment, a two vessel system was used. One vessel contained a malodor mixture (Vessel 1) and one vessel contained fabric treated and untreated with the encapsulated materials (Vessel 2). The systems were connected with tubing so vapor transfer could take place from vessel 1 to vessel 2. The transfer was carried out using a controlled flow suction pump.

A specific amount of liquid malodor mixture (6 ingredients) was initially placed in vessel 1 for a set period of time to allow the headspace of the vessel to equilibrate to saturation.

Vessel 2 contained the test towels some of which were treated with water as the control and some of which were treated with encapsulated neobee oil diluted in water for the sample. The towels used were 100% cotton face cloths and they were sprayed with water or the aqueous encapsulated test mixture and then dried at room temperature for 2 hours before placing in vessel 2.

Once vessel 2 was prepared and vessel 1 reached equilibration the malodor gas was transferred from #1 to #2 using a controlled flow suction pump. The gas flow was then removed from Vessel 2 and trapped on Tenax collection tube (commercially available from SUPELCO) that was inserted between the pump and vessel 2. This tube was used to trap any of the malodor ingredients that were released after flowing through Vessel 2 containing the treated cloth.

This Tenax tube was then desorbed onto a GC/MS for analysis of the malodor released from Vessel 2. The results of these experiments are presented in Table 1.

TABLE 1

| | Ng/L | Ng/L |
|---|---|---|
| Malodor Ingredient | Control | Encap. |
| Total Malodor | 1001 | 395 |
| Pyridine | 627 | 274 |
| Furfural | 82 | 23 |
| Isovaleric acid | 28 | 18 |
| Heptanal | 161 | 44 |
| Hexanoic acid | 17 | 7 |
| Dibutyl sulfide | 86 | 29 |

With the addition of capsules to the fabric, the reduction in malodor released from fabric ranged from 44% for pyridine to 75% for heptanal.

### EXAMPLE 3

### Effectiveness of Capsule Particle Composition Compared to Water, Solvents and Oils Toward Malodor Reduction

An analysis of the malodor present in the headspace of the above treated towels was completed. The experimental protocol for this analysis was that used in Example 2.

TABLE 2

| | | | | | | Ng/L | | | |
|---|---|---|---|---|---|---|---|---|---|
| | | | | | | cap | | Ng/L | |
| Malodor | Ng/L | Ng/L | % | Ng/L | % | wall | % | Encap | % |
| Ingredient | water | Alcohol | removed | oil/alc | removed | mat | removed | oil | removed |
| Total | 1001 | | | | | | | 395 | |
| Malodor | | | | | | | | | |
| Pyridine | 627 | 813 | 0 | 604 | 3.7 | 377 | 40 | 274 | 56 |
| Furfural | 82 | 37 | 55 | 40 | 51 | 44 | 46 | 23 | 72 |
| Isovaleric | 28 | 36 | 0 | 23 | 18 | 8 | 71 | 18 | 36 |
| Acid | | | | | | | | | |
| Heptanal | 161 | 123 | 24 | 116 | 28 | 97 | 40 | 44 | 73 |
| Hexanoic | 17 | 11 | 35 | 14 | 18 | 8 | 53 | 7 | 59 |
| Acid | | | | | | | | | |
| Dibutyl | 86 | 97 | 0 | 108 | 0 | 62 | 28 | 29 | 66 |
| Sulfide | | | | | | | | | |

This experiment shows that most of the benefit toward malodor reduction is provided by the capsule material, although there is some added benefit of a solvent/oil/fragrance added to the capsule.

### EXAMPLE 4

### Comparative Example of the Effectiveness of the Capsule Particle Composition as Compared to a Commercial Malodor Reducing Agent

Sachets were filled with both the encapsulation material and sodium bicarbonate, typically used in many applications with malodor reduction claims. The sachets were added to gallon jars containing a diluted malodor cocktail. One jar contained only malodor, one contained just the sachet material, one contained a sachet with the encapsulation material and the final one contained a sachet with sodium bicarbonate. After equilibration, equivalent amounts of vapor were collected on Tenax tubes. This tube was used to trap any of the malodor ingredients. The tubes were then analyzed by GC/MS and the malodor ingredients in the vapor phase were quantified and are presented in Table 3.

TABLE 3

| | | | | Sachet | | | |
|---|---|---|---|---|---|---|---|
| | Jar w/ | | | w/ | | Sachet | |
| | malodor | Empty | % | sodium | % | w/ encap | % |
| Malodor | only | Sachet | re- | bicarb | re- | material | re- |
| Ingredient | (Ng/L) | (μg/L) | duced | (ng/L) | duced | (ng/L) | duced |
| | 66220 | 40950 | | 34480 | | 26890 | |
| Pyridine | 32020 | 14220 | 56 | 12090 | 62 | 13050 | 59 |
| Furfural | 2780 | 5790 | 0 | 5130 | 0 | 2810 | 0 |
| Isovaleric | 640 | 980 | 0 | 600 | 6 | 690 | 0 |
| acid | | | | | | | |
| Heptanal | 19950 | 11440 | 42 | 9040 | 55 | 4130 | 79 |
| Hexanoic | 2100 | 1420 | 32 | 1140 | 46 | 580 | 72 |
| acid | | | | | | | |
| Dibutyl | 8730 | 7100 | 19 | 6480 | 26 | 5630 | 36 |
| sulfide | | | | | | | |

The encapsulation material was shown to be a more effective malodor reducing agent than even sodium bicarbonate and the overall malodor in the vapor phase was reduced by 60% using a sachet with the encapsulation material.

### EXAMPLE 5

### Effectiveness of the Capsule Materials Toward Malodor Reduction in Fabric Refresher Sprays

Towels were washed in a fabric conditioner and detergent with and without capsules in the formulation and these towels were tested in the same malodor reduction experiment described in Example 3. A reduction in the malodor ingredients observed in the vapor above the towels washed with products with and without capsules is shown in the following Table 4.

TABLE 4

| | | Towels | | Towels | Towels | |
|---|---|---|---|---|---|---|
| | Towels | washed in | | washed in | washed in | |
| | washed in | fabric | | laundry | laundry | |
| | fabric | conditioner | | detergent | detergent | |
| | conditioner | (with | | (no | (with | |
| Malodor | (no caps.) | caps.) | | caps.) | caps.) | |
| Ingredient | (ng/L) | (ng/L) | % reduced | (ng/L) | (ng/L) | % reduced |
| Total | 1004.6 | 874.2 | | 1004 | 441 | |
| Malodor | | | | | | |
| Pyridine | 596 | 602 | 0 | 635 | 309 | 51 |
| Furfural | 103 | 15 | 85 | 82 | 15 | 82 |
| Isovaleric | 17 | 29 | 0 | 28 | 20 | 29 |
| acid | | | | | | |
| Heptanal | 168 | 110 | 35 | 144 | 47 | 67 |
| Hexanoic | 19 | 14 | 26 | 16 | 11 | 31 |
| acid | | | | | | |
| Dibutyl | 100 | 104 | 0 | 98 | 39 | 60 |
| sulfide | | | | | | |
| Indole | 1.6 | 0.2 | 88 | 1.0 | ND | 100 |

ND = not detectable

As the results of this experiment show, the addition of capsules to the fabric conditioner and laundry detergent products does offer a malodor reduction benefit to articles wash with these products. The extent of the benefit from these wash-off products is dependent on the amount of encapsulated material deposited and the manner in which the material is deposited to the fabric.

What is claimed is:

1. An odor-absorbing composition comprising:
an effective amount of a capsule particle composition wherein the capsule particles comprise an active ingredient and an encapsulating polymer.

2. The odor-absorbing composition of claim 1 comprising from about 0.001% to about 10% by weight of capsule particles.

3. The odor-absorbing composition of claim 1 wherein the encapsulating polymer comprises a crosslinked network of polymers comprising a melamine-formaldehyde:acrylamide-acrylic acid copolymer wherein the mole ratio is in the range of from 9:1 to 1:9.

4. The odor-absorbing composition of claim 1 wherein the active material is a fragrance.

5. The odor-absorbing composition of claim 1 wherein the active material is a solvent.

6. The odor-absorbing composition of claim 1 wherein the active material is a mixture of a fragrance and a solvent.

7. A method to reduce the perceived malodor from a surface selected from a hard surface, a soft surface and surrounding vapor phase comprising the steps of providing a capsule particle composition and contacting the capsule particle composition with the malodor and absorbing the malodor with the capsule particle composition.

8. The method of claim 7 comprising from about 0.001% to about 10% by weight of capsule particles.

9. The method according to claim 7 wherein the capsule particle composition comprises an active ingredient and an encapsulating polymer.

10. The method according to claim 7 wherein the active ingredient is selected from the group consisting of solvents, fragrances and mixtures thereof.

11. The method according to claim 7 wherein the encapsulating polymer comprises a crosslinked network of polymers comprising a melamine-formaldehyde:acrylamide-acrylic acid copolymer wherein the mole ratio is in the range of from 9:1 to 1:9.

12. The method according to claim 7 wherein the surface is a hard-surface.

13. The method according to claim 7 wherein the surface is a soft-surface.

14. The method according to claim 7 wherein the surface is the surrounding vapor phase.

15. An article of manufacture comprising the composition of claim 1 in a spray dispenser.

16. The article of manufacture of claim 15 comprising from about 0.001% to about 10% by weight of capsule particles.

17. A wash-off product comprising the odor-absorbing composition of claim 1.

18. The wash-off product of claim 14 wherein the wash-off product is selected from the group consisting of liquid, powder and gel laundry detergent and fabric softeners, liquid, gel and powder dish detergents, automatic dish detergents, hair shampoos and conditioners.

19. The wash-off product of claim 17 comprising from about 0.001% to about 10% by weight of capsule particles.
